(19) United States
(12) Patent Application Publication
(10) Pub. No.: US 2011/0151006 A1
(43) Pub. Date: Jun. 23, 2011

(54) STIMULI-RESPONSIVE HYDROGEL

(76) Inventors: Wilfried Weber, Freiburg im Breisgau (DE); Martin Fussenegger, Magenwil (CH); Ronald Schoenmakers, Rijswijk (NL)

(21) Appl. No.: 12/996,492

(22) PCT Filed: Jun. 5, 2009
(86) PCT No.: PCT/EP2009/004050

§ 371 Date: Dec. 6, 2010

(30) Foreign Application Priority Data
Jun. 6, 2008 (EP) 08010312.0
Nov. 4, 2008 (EP) 08019267.7

(51) Int. Cl. A61K 9/14; A61K 38/18; A61P 5/00

(52) U.S. Cl. 424/487; 424/484; 514/8.1

(57) ABSTRACT

The present invention relates to a hydrogel comprising a polymer, a first polypeptide and a polypeptide binding partner, wherein the polypeptide binding partner is a second polypeptide, a nucleic acid or a small molecule, and wherein the interaction between the first polypeptide and the polypeptide binding partner stabilizes the hydrogel and is modulated by the addition of a modulating compound. A drug may be physically entrapped in the hydrogel, bound to the polymer forming the hydrogel structure, or bound to the first polypeptide or the polypeptide binding partner, and then be set free on addition of the modulating compound. Such a hydrogel comprising a drug may be injected into a patient, and drug release modulated by orally administering the modulating compound.

### FIELD OF THE INVENTION

The invention relates to a hydrogel comprising a polymer, a polypeptide and a polypeptide binding partner, wherein the interaction of the polypeptide with its binding partner can be modulated by a third compound. This hydrogel is especially useful in drug delivery.

### BACKGROUND OF THE INVENTION

Stimuli-sensing hydrogels responsive to temperature, light, calcium, antigens, DNA and specific enzymes hold great promises as smart materials for drug delivery within the body (reviewed in Kopecek J., Eur J Pharm Sci 20, 1-16, 2003), for tissue engineering (Lutolf M. P. and Hubbell J. A., Nat Biotechnol 23, 47-55, 2005) or as (nano-) valves in microfluidic applications (Beebe D. J. et al., Nature 404, 588-90, 2000). Such materials commonly respond to triggers, which are difficult to apply in a patient background in the case of physical stimuli (e.g. light, temperature) or in the case of molecule-based stimuli due to stimulus concentrations hardly achievable in a physiologic background (e.g. antibody concentrations in the g/l range). In contrast, the mode of action for pharmaceutical substances is designed to occur within physiologic limits and therefore, hydrogels based on a pharmacologic mode of action are expected to show high compliance with future therapeutic applications.

### SUMMARY OF THE INVENTION

The present invention relates to a hydrogel comprising a polymer, a first polypeptide and a polypeptide binding partner, wherein the polypeptide binding partner is a second polypeptide, a nucleic acid or a small molecule, and wherein the interaction between the first polypeptide and the polypeptide binding partner is non-covalent and modulated by the addition or withdrawal of a modulating compound.

In particular the invention relates to such a hydrogel, wherein the first polypeptide and the polypeptide binding partner are linked to the polymer, and wherein the interaction between the first polypeptide and the polypeptide binding partner stabilizes the hydrogel.

In particular, the invention relates to a hydrogel, wherein the first polypeptide and/or the polypeptide binding partner are covalently linked to the polymer, or linked to the polymer by a strong, specific non-covalent linkage.

Either the first polypeptide or the polypeptide binding partner may be linked to the polymer, and the corresponding polypeptide binding partner or the first polypeptide, respectively, linked to a compound of interest, for example a drug. Alternatively, the compound of interest may be physically entrapped in the hydrogel, or bound to the polymer forming the hydrogel.

The interaction between the first polypeptide and the polypeptide binding partner is cleaved by the addition or withdrawal of a modulating compound.

The invention furthermore relates to a system of drug delivery comprising the hydrogel, wherein either the first polypeptide or the polypeptide binding partner are linked to the polymer, and the corresponding polypeptide binding partner or the first polypeptide, respectively, linked to a drug, and further comprising a compound cleaving the interaction between the first polypeptide and the polypeptide binding partner.

Likewise the invention relates to a system of drug delivery comprising the hydrogel, wherein the first polypeptide and/or the polypeptide binding partner are linked to the polymer and a drug is physically entrapped in the hydrogel structure stabilized by the interaction between the first polypeptide and the polypeptide binding partner, and further comprising a compound cleaving the interaction between the first polypeptide and the polypeptide binding partner thereby loosening the hydrogel structure to set free said drug. In a variation of this principle, the drug may be bound to the polymer forming the hydrogel. On addition of the compound cleaving the interaction between the first polypeptide and the polypeptide binding partner, the hydrogel breaks down and a drug-polymer complex is set free.

Accordingly, the invention relates also to a method of delivering a drug to a patient in need thereof, wherein a hydrogel is administered to the patient, wherein either the first polypeptide or the polypeptide binding partner are linked to the polymer, and the corresponding polypeptide binding partner or the first polypeptide, respectively, are linked to the drug, and after the hydrogel has reached its intended site of action the compound cleaving the interaction between the first polypeptide and the polypeptide binding partner is administered.

Likewise, the invention relates to a method of delivering a drug to a patient in need thereof, wherein a hydrogel is administered to the patient, wherein the first polypeptide and/or the polypeptide binding partner are linked to the polymer and a drug is physically entrapped in the hydrogel structure or bound to the polymer forming the hydrogel structure stabilized by the interaction between the first polypeptide and the polypeptide binding partner, and after the hydrogel has reached its intended site of action the compound cleaving the interaction between the first polypeptide and the polypeptide binding partner is administered thereby loosening the hydrogel structure to set free said drug, or the drug bound to the polymer, respectively.

### BRIEF DESCRIPTION OF THE FIGURES

FIG. 1: Pharmacologically-triggered hydrogel

(a) Bacterial gyrase subunit B (GyrB) coupled to a polyacrylamide backbone is dimerized by coumermycin (+C) resulting in gelation of the hydrogel. In the presence of novobiocin (+N) GyrB is dissociated resulting in dissolution of the hydrogel.

(b) Coupling of proteins to the polyacrylamide backbone. Polyacrylamide is functionalized with nitrilotriacetic acid chelating a Ni²⁺ ion to which GyrB can bind via a hexahistidine sequence.

FIG. 2: Design of pharmacologically-controlled hydrogels

(a) Antibiotic-dependent hydrogel formation. Hexahistidine-tagged GyrB is incubated in the presence of coumermycin (+C, GyrB:coumermycin=2:1, mol/mol), novobiocin (+N, GyrB:novobiocin=1:10, mol/mol) or in the absence of any antibiotic (w/o). The GyrB complexes are mixed with Ni²⁺-charged poly(AAM-co-NTA-AAM) and the resulting viscous structures are incubated in PBS for 12 h prior to quantification of GyrB protein released into the buffer.

(b) GyrB dimerization-specific hydrogel. GyrB is dimerized by coumermycin (GyrB:coumermycin=2:1) and further incubated in the presence (+DMS) or absence (−DMS) of the amine-specific bifunctional crosslinker dimethyl suberimidate (DMS) for covalently stabilizing the GyrB dimers. GyrB dimers are mixed with Ni²⁺-charged poly(AAM-co-NTA-AAM) resulting in formation of the hydrogel. Following swelling over night in PBS, the hydrogels are placed in PBS containing 1 mM novobiocin and polymer dissolution is monitored by quantifying the released GyrB protein into the buffer.

FIG. 3: Adjustable pharmacologically triggered disintegration of the hydrogel

Hydrogels are incubated in PBS in the presence of different novobiocin concentrations (0-1 mM) and hydrogel disintegration is measured by quantification of GyrB released into the buffer.

FIG. 4: Human vascular endothelial growth factor 121 (VEGF₁₂₁) release

VEGF₁₂₁ is incorporated into the hydrogel and incubated in the presence of increasing novobiocin concentrations. VEGF₁₂₁ release into the buffer is followed over time.

FIG. 5: Novobiocin-induced swelling of the hydrogel

Hydrogels incorporating partially chemically crosslinked GyrB units are incubated in the presence or absence of 1 mM novobiocin while monitoring changes in polymer size.

### DETAILED DESCRIPTION OF THE INVENTION

The present invention relates to a hydrogel comprising a polymer, a first polypeptide and a polypeptide binding partner, wherein the polypeptide binding partner is a second polypeptide, a nucleic acid or a small molecule, and wherein the interaction between the first polypeptide and the polypeptide binding partner is non-covalent and modulated by the addition or withdrawal of a modulating compound.

Suitable polymers are, for example, poly-vinyl-based polymers like polyacrylamide, polyethylene glycol, poly-dimethyl-diallyl-ammonium chloride and N-(2-hydroxypropyl)-methacrylamide, polypeptides like fibrin, collagen and poly-L-lysine, and poly-carbohydrates like alginate, optionally modified celluloses, e.g. cellulose, hydroxyethyl-cellulose (HEC), hydroxypropylcellulose (HPC) and hydroxypropylmethylcellulose (HPMC), dextran and starch. Preferred polymers are, for example, polyethylene glycol, polyacrylamide and fibrin. Most preferred as the polymer is polyethylene glycol. Suitable polymers may be modified by reaction with further polymeric compounds, e.g. to fine-tune solubility.

A “modulating compound” as used herein is a compound breaking or causing the non-covalent interaction between the first polypeptide and the polypeptide binding partner under physiological conditions. It is understood that a protein denaturing compound destroying tertiary and secondary structures of polypeptides, e.g. inorganic salts and acids, organic solvents such as methanol, ethanol or acetone, organic acids such as acetic acid, trichloroacetic acid, picric acid or sulfosalicylic acid, chaotropic agents such as urea or guanidinium salts, disulfide bond reducers such as 2-mercaptoethanol, dithiothreitol or tris(2-carboxyethyl)phosphine, and related compounds are not considered a modulating compound in the sense of the invention. Under “physiological conditions” in the sense of the invention it is understood that the compound is breaking or causing the non-covalent interaction between the first polypeptide and the polypeptide binding partners at a concentration below 1 mg/ml.

Pairs of a first polypeptide and a polypeptide binding partner, wherein the polypeptide binding partner is a second polypeptide, are for example, GyrB-GyrB (gyrase subunit B), FKBP-FRB (FK-binding protein-a domain (FRB) of the lipid kinase protein homologue FRAP (FKBP-rapamycin-associated protein)), F_{M}-F_{M} (F36M mutation of FK-binding protein), ToxT-ToxT (ToxT Protein of V. cholerae), DHFR-DHFR (dihydrofolate reductase), FKBP-FKBP (FK-binding protein), FKBP-Cyp (FK-binding protein-cyclophilin) and Cyp-Cyp (Cyclophilin). The first polypeptide and/or the polypeptide binding partners may as well be homomultimers of the above-listed polypeptides or heteromultimers between at least two of the above-listed polypeptides. The first polypeptide and/or the polypeptide binding partner may further be covalently linked to a further polymer, e.g. polyethylene glycol or polyacrylamide, in order to influence solubility and to prevent aggregation. The corresponding modulating compound influencing the interaction between the polypeptides either by addition or withdrawal are, for example, coumarin antibiotics (for GyrB-GyrB), rapamycin or FK506 and derivatives (e.g. rapalogs, mTOR inhibitors) (for FKBP-FRB and F_{M}), cyclosporins and derivatives (for Cyp), FK506 (for FKBP-FRB and F_{M}), virtstatin (for ToxT), and methotrexate and derivatives thereof (e.g. antifolates) (for DHFR-DHFR). Preferred as modulating compounds are small organic compounds, for example compounds of a molecular weight between 100 and 5000, in particular between 100 and 2000.

In a particular example, the first polypeptide and the polypeptide binding partner are the same compound having a tendency to form dimers. Particular examples of such dimerizing polypeptides are GyrB, F_{M}, ToxT, FKBP, and DHFR. The hydrogel comprising such dimerizing polypeptide may further contain a compound inducing dimerization. For example such a compound inducing dimerization are coumarin antibiotics, rapamycin and derivatives, virstatin, FK1012, and methotrexate and derivatives thereof. This dimerizing compound may fall under the definition of a modulating compound as set forth herein-above or hereinbelow. Alternatively the modulating compound influencing the interaction between the first polypeptide and the polypeptide binding partner may be a modulating compound neutralizing the activity of the dimerizing compound, and by this neutralization lead to substantial reduction of the interaction of the dimerizing polypeptide. Such compounds which neutralize the dimerizing effect are, for example, the same compounds mentioned above to be dimerizing compounds when used in a substantial excess, or preferably other, different representatives of the same class of dimerizing compounds, e.g. the class of coumarin antibiotics, rapamycin and derivatives, and methotrexate, antifolates and derivatives thereof, and also FK506.

Pairs of a first polypeptide and a polypeptide binding partner, wherein the polypeptide binding partner is a nucleic acid, are for example, E-ETR (MphR(A) protein and its operator ETR of E. coli), PIP-PIR (PIP protein of Streptomyces pristinaespiralis and its operator PIR), TetR-tetO (Tn10-derived tetracycline repressor TetR and its operator tetO), ArgR-argO (arginine-responsive repressor and its operator argO), ArsR-arsO (arsenic-responsive repressor and its operator arsO), and HucR-hucO (uric acid-responsive repressor and its operator hucO). Other such pairs are the ones described by Ramos J. L. et al. (Microbiol Mol Biol Rev 69, 326-56, 2005), Martinez-Bueno M. et al. (Bioinformatics 20, 2787-91, 2004), and the ones that are listed in the database BacT regulators (http://www.bactregulators.org/). The corresponding modulating compounds influencing the interaction between the polypeptides either by addition or withdrawal are, for example, macrolide antibiotics (for E-ETR), streptogramin antibiotics (for PIP-PIR), tetracycline antibiotics (for TetR-tetO), arginine (for ArgR-argO), heavy metals (for ArsR-arsO), and uric acid (for HucR-hucO).

Pairs of a first polypeptide and a polypeptide binding partner, wherein the polypeptide binding partner is a small molecule, are, for example, GyrB-coumarin antibiotics, FKBP-mTOR inhibitors, FRB-mTOR inhibitors, F_{M}-mTOR inhibitors, Cyp-cyclosporins, Cyp-ascomycins, DHFR-antifolate, streptavidin-biotin analog, avidin-biotin analog, neutravidin-biotin analog, steroid hormone receptors-steroid hormones and analogs thereof, and ToxT-virstatin.

In the case, where the polypeptide binding partner is a small molecule, the polypeptide binding partner has a molecular weight of preferably <5000 g/mol, in particular between 100 and 5000 g/mol.

Coumarin and aminocoumarin antibiotics include, for example, novobiocin, chlorobiocin, coumermycin and dihydronovobiocin.

A cyclosporin or an ascomycin can be, for example, Cyclosporin A (NEORAL®), ISAtx-247, FK506 (tacrolimus), FK778, ABT-281 or ASM981.

An mTOR inhibitor can be, for example, rapamycin or a derivative thereof, e.g. Sirolimus (RAPAMUNE®), Deforolimus, Temsirolimus, Zotarolimus, Everolimus (Certican®), CCI779, ABT578, biolimus-7, biolimus-9, a rapalog, e.g. AP23573, azathioprine, campath 1H, a S1P receptor modulator, e.g. FTY720, or an analogue thereof.

Rapalogs include, among others, variants of rapamycin having one or more of the following modifications relative to rapamycin: demethylation, elimination or replacement of the methoxy group at C7, C42 and/or C29; elimination, derivatization or replacement of the hydroxy group at C13, C43 and/or C28; reduction, elimination or derivatization of the ketone function at C14, C24 and/or C30; replacement of the 6-membered pipecolate ring with a 5-membered prolinyl ring; and alternative substitution on the cyclohexyl ring or replacement of the cyclohexyl ring with a substituted cyclopentyl ring. Further modifications considered are presented in the background sections of U.S. Pat. Nos. 5,525,610; 5,310,903 and 5,362,718, and also in U.S. Pat. No. 5,527,907. Further considered is selective epimerization of the C28 hydroxy group (WO 01/14387). Further considered is the use of rapamycin analogs containing various phosphorus-containing moieties, such as described in WO 03/064383 and WO 05/16252. Other rapalogs considered are described in U.S. Pat. No. 6,984,635, U.S. Pat. No. 6,649,595 and U.S. Pat. No. 7,091,213.

Antifolates include, for example, compounds binding to DHFR like, for example, methotrexate, trimethoprim, diaminopyrimidines like brodimoprim and epiroprim, or iclaprim. Other DHFR inhibitors considered are those described in Hawser S. et al., Biochemical Pharmacology 71, 941-948, 2006.

Biotin analogs include, for example, compounds binding to streptavidin, neutravidin or avidin like, for example, biotin, HABA, desthiobiotin, iminobiotin or diaminobiotin.

The above-listed small molecule polypeptide binding partners may be subjected to derivatization suitable for binding to the polymer or another compound of interest. Such derivatization may include the introduction of an amine, an amide, a thiol, a hydroxyl, an aldehyde, an azide, an alkine, a ketone, an epoxide or a carboxy function.

Particular preferred pairs of a first polypeptide and a polypeptide binding partner, together with the corresponding modulating compound aminocoumarin antibiotics (e.g. coumermycin and novobiocin for GyrB-GyrB), rapamycin, FK506 and its derivatives AP21998 and AP22542 (for F_{M}-F_{M} and FKBP-FRB) are the combinations GyrB-GyrB, F_{M}-F_{M} and FKBP-FRB.

Most preferred are GyrB-GyrB and F_{M}-F_{M}.

In particular, the invention relates to such a hydrogel, wherein the first polypeptide and the polypeptide binding partner are linked to the polymer, and wherein the interaction between the first polypeptide and the polypeptide binding partner stabilizes the hydrogel. For example, the combination of GyrB with coumermycin is particularly useful for stabilizing a polymer.

In particular, the invention relates to such a hydrogel, wherein the first polypeptide and/or the polypeptide binding partner are covalently linked to the polymer, or linked to the polymer by a strong, specific non-covalent linkage. A strong non-covalent linkage in the sense of the present invention is a linkage with a dissociation constant of below 10⁻⁵ M under physiological conditions. The polypeptide and its binding partner can be coupled to the polymer by specific linkers like, for example, chelate-forming entities like NTA and polyhistidine binding to a multivalent metal ion, peptide bonds, thiols coupled to maleimide or vinylsulfones, a halotag (Los G. V. et al., Methods Mol. Biol. 356, 195-208, 2007), a SNAP-tag or a CLIP-tag (Gautier A. et al., Chem. Biol. 15, 128-36, 2008) or by a transglutaminase reaction bond (Ehrbar M. et al., Biomaterials 29, 1720-9, 2008). Such hydrogels are usually prepared by coupling the first polypeptide and the polypeptide binding partner to the polymer backbone, mixing both reaction products, and varying the concentration of the modulating compound in a way that the first polypeptide and the polypeptide binding partner interact with each other thereby forming a hydrogel. When changing the concentration of the modulating compound or adding a second modulating compound neutralizing the effect of the first one, the rigid structure is broken up and the hydrogel reverts to a substantially less rigid structure, e.g. a hydrosol.

For further stabilizing the hydrogel, additional cross-links can be introduced by chemically crosslinking the polymer backbone or crosslinking the first polypeptide with the polypeptide binding partner. Suitable crosslinkers are any homo- or heterofunctional compounds showing at least two sites for binding to another molecule like the ones described in Bioconjugate Techniques (2^{nd} Edition by Greg T. Hermanson, Academic Press, 2008).

Additionally, semi-interpenetrating polymer networks (semi-IPN, meaning a polymer network of two or more polymers wherein at least one polymer is crosslinked and at least one polymer is not crosslinked, as described for example in Miyata T. et al., Nature 399, 766-769, 1999) containing the first polypeptide and the polypeptide binding partner are as well within the scope of the invention

Either the first polypeptide or the polypeptide binding partner may be linked to the polymer, and the corresponding polypeptide binding partner or the first polypeptide, respectively, linked to a compound of interest, for example a drug.

A compound of interest is any substance with a beneficial effect on the host into which the hydrogel has been implanted.

The drug may be any drug selected from the classes of cytostatic and cytotoxic drugs, antibiotics, antiviral drugs, anti-inflammatory drugs, growth factors, cytokines, hormones, antibodies, pain-relievers, polynucleic acids like siRNA, miRNA, DNA and viral particles. Preferred drugs are those that cannot be administered orally, like polypeptide-based drugs.

In particular the drug is a drug which, to exert its full potential, has first to be transported to the site of action. Examples are monoclonal antibodies, growth factors and cytokines.

The drug may either be physically entrapped in the hydrogel structure or bound to the polymer forming the hydrogel, and might be released thereof as the free drug or as drug-polymer complex, respectively, by dissolution or swelling of the hydrogel induced by addition or withdrawal of the modulating compound, breaking the hydrogel structure stabilized by the interaction between polypeptide and the polypeptide binding partner. Alternatively, the drug is bound to the polypeptide binding partner, whereas the polypeptide binding partner is bound in the hydrogel to the first polypeptide. Addition or withdrawal of the modulating compound will cleave the interaction between the first polypeptide and the polypeptide binding partner thereby liberating the drug bound to the polypeptide binding partner. The reverse configuration, where the drug is bound to the first polypeptide and the polypeptide binding partner is immobilized in the hydrogel, is as well within the scope of this invention.

The invention furthermore relates to a system of drug delivery comprising the hydrogel wherein either the first polypeptide or the polypeptide binding partner are linked to the polymer, and the corresponding polypeptide binding partner or the first polypeptide, respectively, linked to a drug, and further comprising a compound cleaving the interaction between the first polypeptide and the polypeptide binding partner.

Preferred components of such a drug delivery system are those hydrogels mentioned above as being preferred, e.g. comprising a preferred polymer, preferred combinations of a first polypeptide and its polypeptide binding partner, and further the suitably adapted preferred modulating compound. Particular situations when such a hydrogel is preferably used are, for example, when the drug must be administered repeatedly or over a longer time frame. For example, the hydrogel may be applied by injection to the particular site of action, and the modulating compound might be applied orally, so that the modulating compound will diffuse to the site where the hydrogel has been injected, so that it will modulate the properties of the hydrogel (swelling or dissolution) and the liberation of the drug. Alternatively, hydrogels might be designed responsive to endogenous modulating compounds like uric acid so that a chance of the physiological concentrations of the endogenous compound will modify the properties of the gel and will modulate the liberation of the embedded drug.

Accordingly, the invention relates also to a method of delivering a drug to a patient in need thereof, wherein a hydrogel is administered to a patient, wherein either the first polypeptide or the polypeptide binding partner are linked to the polymer, and the corresponding polypeptide binding partner or the first polypeptide, respectively, are linked to the drug, and after the hydrogel has reached its intended site of action the compound cleaving the interaction between the first polypeptide and the polypeptide binding partner is administered.

Likewise, the invention relates to a method of delivering a drug to a patient in need thereof, wherein a hydrogel is administered to the patient, wherein either the first polypeptide or the polypeptide binding partner are linked to the polymer and a drug is physically entrapped in the hydrogel structure or bound to the polymer forming the hydrogel structure stabilized by the interaction between polypeptide and the polypeptide binding partner, and after the hydrogel has reached its intended site of action the compound cleaving the interaction between the first polypeptide and the polypeptide binding partner is administered thereby loosening the hydrogel structure to set free said drug or drug-polymer complex, respectively.

Compared with a normal application of the drug to the patient the present method is much more convenient since the drug-comprising hydrogel must only be administered once by injection into the patient, and the drug can be released thereof on demand by taking an orally-available modulating compound. Thus repeated injections are replaced by one injection and some orally active compound in the form of a tablet, capsule, pill, or the like.

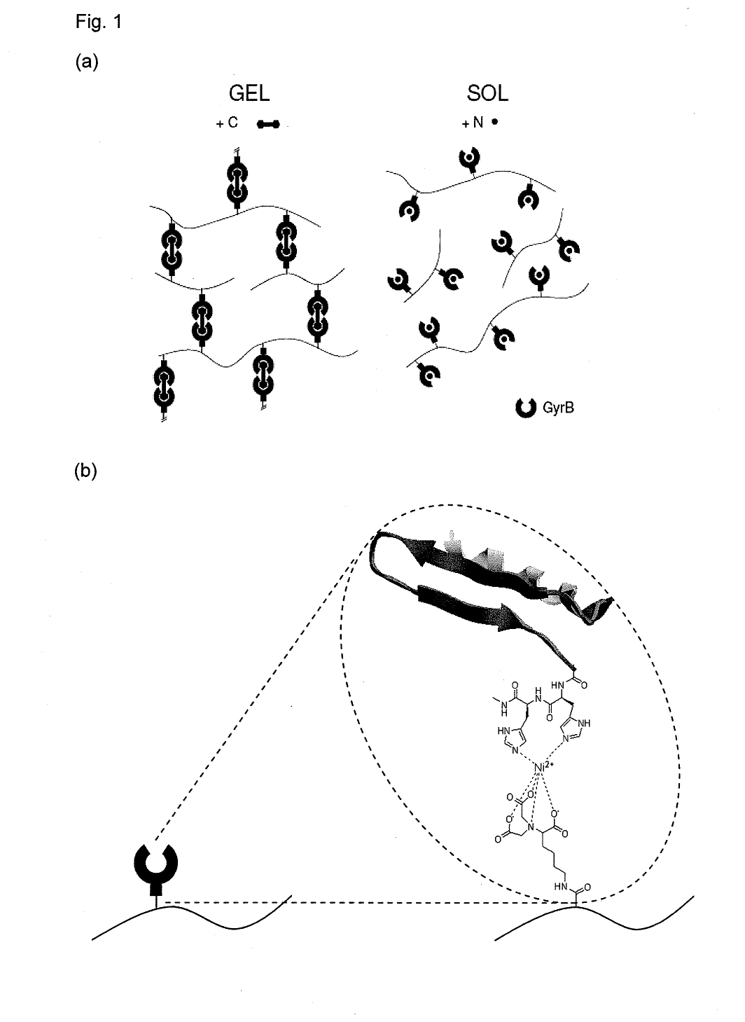

A particular hydrogel according to the invention is the antibiotic-responsive gel based on polyacrylamide grafted with bacterial gyrase subunit B (GyrB), which can be dimerized by the aminocoumarin antibiotic coumermycin, thereby resulting in gelation and three-dimensional stabilization of the hydrogel (FIG. 1a). Upon addition of the aminocoumarin novobiocin (Albamacin®), the interaction between GyrB and coumermycin is competitively inhibited, the three-dimensional structure is loosened and the hydrogel changes to the solstate (FIG. 1a). In the particular example, the polymer backbone is polyacrylamide functionalized with nitrilotriacetic acid for chelating Ni²⁺ ions to bind hexahistidine-tagged (His₆) GyrB (FIG. 1b). For construction of the polymer backbone, 2,2′-(5-acrylamido-1-carboxypentylazanediyl)diacetic acid (NTA-AAm) is synthesized, co-polymerized with acrylamide (AAm), and the NTA groups are charged with Ni²⁺. The resulting polymer poly(AAm-co-Ni²⁺-NTA-AAm) has a molecular mass of 42 kDa as judged from size exclusion chromatography with one NTA-AAm group per four acrylamide monomers as deduced from ¹H NMR analysis and reflecting the stoichiometry in synthesis.

The gene for E. coli gyrase subunit B (gyrB) is tagged with the coding sequence for six histidine residues. The coding region is placed under the control of the phage T₇-derived promoter and expressed in E. coli as a soluble cytoplasmatic protein. GyrB is purified via the hexahistidine tag using Ni²⁺-based affinity chromatography. Coumermycin-induced dimerization of genetically engineered GyrB is evaluated by incubating the protein in the presence or absence of coumermycin (GyrB:coumermycin=2:1, mol/mol) with subsequent addition of the amine-specific bifunctional crosslinking agent dimethyl-suberimidate (DMS) and analysis of the complexes on denaturing polyacrylamide gel electrophoresis. In the absence of coumermycin, GyrB migrates predominantly at its predicted size of 27 kDa, whereas addition of the dimerizing antibiotic results in substantial dimer formation migrating at the predicted size of 54 kDa. In order to exclude that the remaining band migrating at 27 kDa in the presence of coumermycin results from inefficient antibiotic-mediated GyrB dimerization but rather from incomplete DMS-mediated covalent crosslinking, ultrafiltration experiments are performed. GyrB is incubated in the presence or absence of coumermycin and subjected to ultrafiltration using a 50 kDa molecular weight cut-off filter. GyrB in the absence of coumermycin passes the filter efficiently (54% of protein in filtrate), whereas only background GyrB levels can be detected in the filtrate, when coumermycin-dimerized GyrB is loaded (2.8% of protein in filtrate) indicating that coumermycin-mediated GyrB dimerization is quantitative.

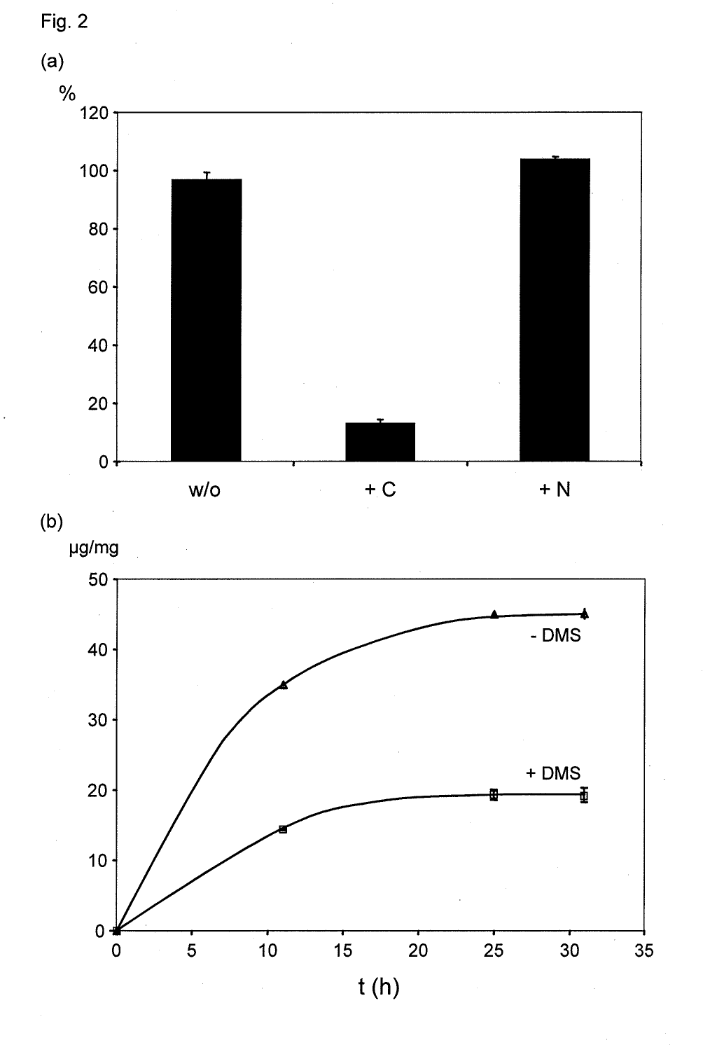

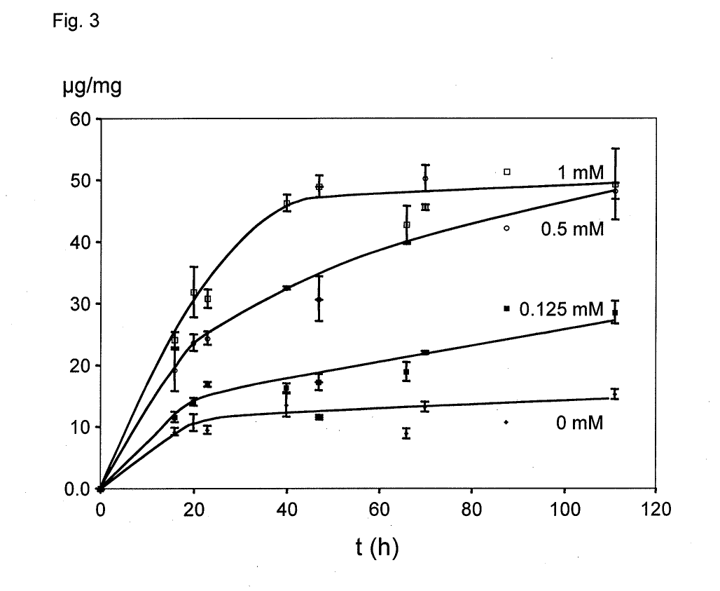

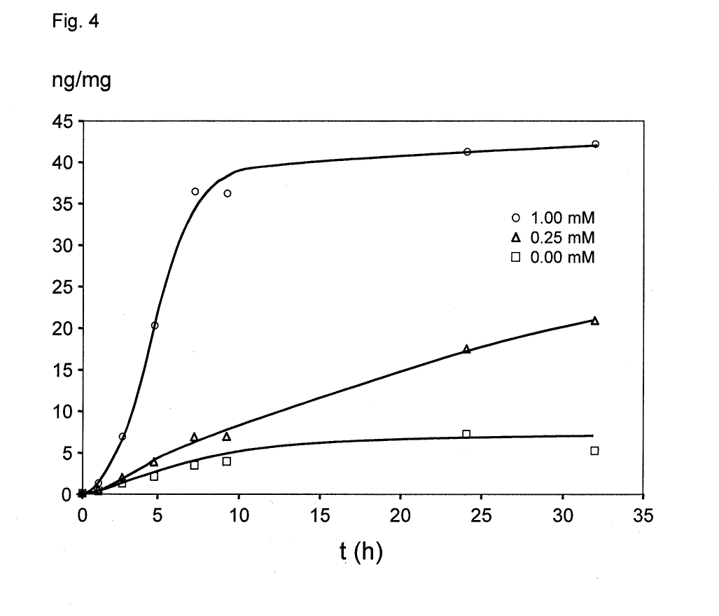

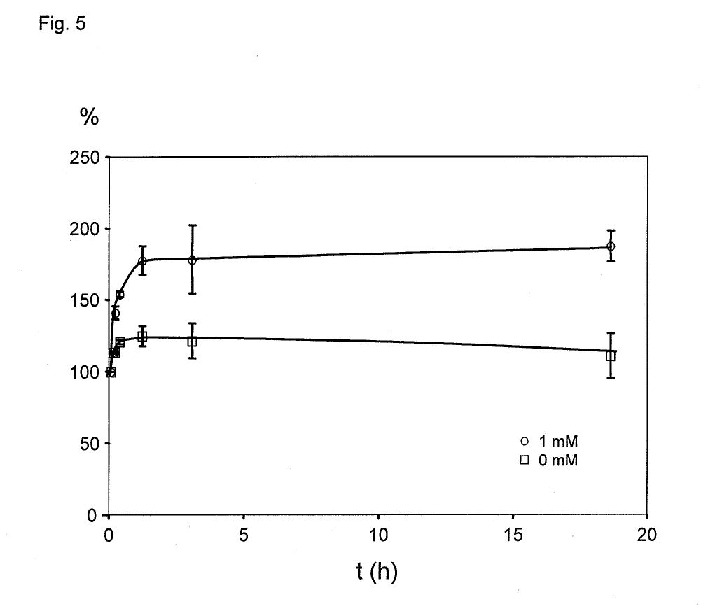

Synthesis of coumermycin-crosslinked hydrogels is validated by incubating hexahistidine-tagged GyrB in the absence or presence of coumermycin or with a ten-fold molar excess of novobiocin. The protein is subsequently mixed with poly(AAm-co-Ni²⁺-NTA-AAm) at a ratio of one GyrB per 11 Ni²⁺ ions chelated in the polymer backbone. The solutions which all become viscous are incubated in PBS for 12 hours prior to quantification of GyrB-polymer complexes released into the buffer (FIG. 2a). In the absence of coumermycin or in the presence of novobiocin, the viscous structures are completely dissolved and GyrB is quantitatively retrieved in the buffer. However, in the presence of coumermycin, GyrB-polymer release is significantly reduced and a hydrogel can be observed thereby indicating the gelling effect of this dimerizing antibiotic (FIG. 2a). In order to demonstrate that the hydrogel formation is effectively due to coumermycin-mediated GyrB dimerization, hydrogels using coumermycin-dimerized GyrB as above or coumermycin-dimerized Gyr which had further been covalently crosslinked by DMS, are synthesized. Following swelling for 12 hours in PBS, the hydrogels are incubated in PBS containing 1 mM novobiocin and hydrogel dissolution is monitored by the release of GyrB-polymer complexes into the buffer (FIG. 2b). While coumermycin-crosslinked hydrogels are dissolved after 11 hours in the presence of novobiocin, hydrogels with DMS-crosslinked GyrB (+DMS) are stable for the observation period of 31 hours (FIG. 2b). This observation confirms that the hydrogel is effectively formed by coumermycin-mediated dimerization of GyrB, which can be reversed by excess novobiocin. Specificity is further demonstrated by addition of antibiotics from other classes (e.g. β-lactams, macrolides), where no impact on gel dissolution can be observed.

Pharmacologically-triggered hydrogel formation and dissolution opens new perspectives for optimal delivery of protein-based pharmaceuticals within the body, provided that the dissolution kinetics of the hydrogel and the release properties of the biopharmaceutical can optimally be adjusted into the therapeutic window. In order to investigate adjustable hydrogel characteristics, coumermycin-dimerized hydrogel is incubated in the presence of increasing novobiocin concentrations, and gel dissolution followed by quantification of released GyrB-polymer complexes (FIG. 3). In the presence of 1 mM novobiocin, the hydrogel dissolves rapidly whereas lower novobiocin concentrations correlate with slower hydrogel dissolution and slower GyrB release demonstrating adjustable dissolution and release kinetics (FIG. 3). The hydrogel is stable for 24 days in the absence of novobiocin. Addition of 1 mM novobiocin at day 24 results in dissolution of the hydrogel until day 26 demonstrating the long-term functionality of the hydrogel.

In order to demonstrate pharmacologically-triggered release of a therapeutic protein from the stimuli-sensing hydrogel, human vascular endothelial growth factors 121 (VEGF₁₂₁). VEGF₁₂₁ is incorporated into the hydrogel (GyrB:VEGF₁₂₁=1000:1, mol/mol) and incubated in the presence of increasing novobiocin concentrations (FIG. 4). In the presence of 1 mM novobiocin, VEGF₁₂₁ is completely released within 10 hours, while only background VEGF₁₂₁ levels are observed in the absence of the stimulus. At intermediate novobiocin concentrations (0.25 mM) VEGF₁₂₁ release kinetics are slower, thereby demonstrating the trigger-adjustable growth factor release characteristics.

The GyrB-based system can as well be used to design hydrogels that swell in the presence of novobiocin. Therefore, hydrogels are prepared as described above with the modification that the coumermycin-dimerized GyrB molecules are chemically crosslinked by equimolar amounts of dimethyl suberimidate. When such gels are incubated in the presence of novobiocin, swelling can be observed (FIG. 5).

### EXAMPLES

### Example 1

### Production of Hexahistidine-Tagged GyrB

Bacterial gyrase subunit B gene (gyrB) is amplified from E. coli DH5α chromosomal DNA using oligonucleotides OWW866 (5′-ggtacttgcacatatgtcgaattcttatgactcctccagtatc-3′, SEQ ID NO:1) and OWW867 (5′-ccagttacaagcttatggtgatggtgatgatggccttcatagtg-3′, SEQ ID NO:2) and ligated (NdeI/HindIII) into pWW301 (Weber C. C. et al., Biotechnol Bioeng 89, 9-17, 2005) thereby placing gyrB under the control of the phage T₇ promoter. pWW873 is transformed into E. coli BL21 START™ (DE3) (Invitrogen, Carlsbad, Calif., cat. no. C601003) and protein production is induced at OD₆₀₀=1 by 1 mM IPTG for 3 h at 37° C. The cell pellet is resuspended in PBS (40 ml per 1000 ml initial culture volume, 50 mM NaH₂PO₄, 300 mM NaCl, 10 mM imidazole, pH 8.0), disrupted using a French press (Thermo Fisher Scientific, Waltham, Mass.), and cell debris are eliminated by centrifugation at 15,000×g for 20 min. The cleared cell lysate is loaded onto an NTA-agarose Superflow column (Qiagen, Hilden, Germany, cat. no. 30210), which is subsequently washed with 10 column volumes PBS, 10 column volumes wash buffer (50 mM NaH₂PO₄, 300 mM NaCl, 20 mM imidazole, pH 8.0) and eluted with 2 column volumes elution buffer (50 mM NaH₂PO₄, 300 mM NaCl, 250 mM imidazole, pH 8.0). The elution buffer is exchanged to PBS by ultrafiltration (10 kDa MW cut-off, Sartorius, Göttingen, Germany, cat. no. VS0202), and GyrB is concentrated to 80 mg/ml.

### Example 2

### Production of VEGF₁₂₁

The expression vector pRSET-VEGF₁₂₁ (Ehrbar M. et al., Circ Res 94, 1124-32, 2004) for hexahistidine-tagged human vascular endothelial growth factor 121 (VEGF₁₂₁) is transformed in E. coli BL21 START™ (DE3), and protein production is induced at OD₆₀₀=0.8 by addition of 1 mM IPTG for 4 h at 37° C. The cell pellet is resuspended in 50 mM Tris/HCl, 2 mM EDTA, pH 8.0 (100 ml per 1000 ml initial culture volume), the cells are disrupted using a French press and the inclusion bodies are pelleted by centrifugation at 15′000×g for 20 min at 4° C. Inclusion bodies are dissolved over night at 4° C. in 6 M urea, 500 mM NaCl, 5 mM imidazole, 20 mM Tris/HCl, pH 7.9 and separated from the cell debris by centrifugation (15′000×g, 30 min, 4° C.). The supernatant is loaded onto an NTA-agarose Superflow column following washing (15 column volumes 6 M urea, 500 mM NaCl, 20 mM imidazole, 20 mM Tris/HCl, pH 7.9) and elution (6 M urea, 500 mM NaCl, 250 mM imidazole, 20 mM Tris/HCl, pH 7.9). The eluate is incubated for 30 min at 22° C. with 2 mM DTT and 2 mM EDTA to reduce disulfide bonds followed by a three step dialysis (3.5 kDa MW cut-off, Pierce, Rockford, Ill., cat. no. 68035): 2×1 h in 4 M urea, 1 mM EDTA, 20 mM Tris/HCl, pH 7.5; 2×1 h in 3 M urea, 1 mM EDTA, 20 mM Tris/HCl, pH 7.5; 1 h in 2 M urea, 150 mM NaCl, 20 mM Tris/HCl, pH 7.5 and over night in 2 M urea, 150 mM NaCl, 20 mM Tris/HCl, pH 7.5. Purified VEGF₁₂₁ is stored at −80° C. VEGF₁₂₁ is quantified using a sandwich ELISA (Peprotech, Hamburg, Germany, cat. no. 900-K10).

### Example 3

### Characterization of GyrB

Concentration of GyrB is analyzed by the Bradford method (Biorad, Muünchen, Germany, cat. no. 500-0006) using BSA as standard. For SDS-PAGE analysis, 12% reducing gels are used with subsequent Coomassie staining. Antibiotic-triggered dimerization of GyrB is analyzed by incubating GyrB in the presence coumermycin A1 (GyrB:coumermycin=2:1, mol/mol; Sigma, St. Louis, Mo., cat. no. C9270) for 30 min at room temperature following covalent crosslinking of the dimers by addition of dimethyl suberimidate×2HCl (DMS, SigmaAldrich, St. Louis, Mo., cat. no. 179523) at a 10-fold molar excess with respect to GyrB for 60 min at room temperature. The dimers are analyzed on SDS-PAGE. For ultrafiltration studies of the coumermycin-induced dimerization, 100 nmol GyrB in 1 ml PBS are incubated with or without 50 nmol coumermycin for 30 min at room temperature. Following addition of 4 ml PBS, the solution is subjected to ultrafiltration (50 kDa MW cut-off, Filtron, Northborough, Mass., cat. no. OD050C36) at 5000×g for 45 min. The retentate (1 ml) is diluted with 5 ml PBS and ultrafiltrated again prior to quantification of the GyrB concentration in the pooled filtrate and retentate.

### Example 4

### Synthesis of 2,2′-(5-acrylamido-1-carboxypentylazanediyl)diacetic acid

### (NTA-AAm)

3.3 mmol acryloyl chloride (ABCR, Karlsruhe, Germany, cat. no. AB172729) dissolved in 15 ml toluene are dropwise added during 4 h to an ice-cooled solution of 3 mmol N,N-bis(carboxymethyl)-L-lysine (Fluka, Buchs, Switzerland, cat. no. 14580) dissolved in 27 ml 0.44 M NaOH. The toluene is evaporated in vacuo and sodium ions are removed with Dowex® 50WX8 (Acros, Geel, Belgium, cat. no. 335351000) prior to lyophilization resulting in a viscous oil (yield: 50%).

### Example 5

### Synthesis of poly(AAm-co-NTA-AAm)

1.5 mmol NTA-AAm and 6.4 mmol acrylamide (AAm, Pharmacia Biotech, Uppsala, Sweden, cat. no. 17-1300-01) are dissolved in 48 ml 50 mM Tris/HCl, pH 8.5 under nitrogen, and polymerization is initiated by addition of 150 μl ammonium peroxodisulphate (APS, 10%, w/v) and 24 μl N,N,N′,N′-tetramethylethylenediamine (TEMED) for 20 h at room temperature. The polymer is concentrated to 20 ml in vacuo and subsequently dialyzed twice (3.5 kDa MW cut-off, Pierce, Rockford, Ill., cat. no. 68035) against 2 L H₂O for 12 h to eliminate salts and toxic low molecular weight compounds like residual acrylamide. The obtained molar ratio of AAm to NTA-AAm is 4 to 1 as determined by ¹H NMR (Avance 500 Bruker BioSpin AG Fällanden, Switzerland). The dialysate is supplemented with 3.5 mmol NiSO₄ and dialyzed twice against 0.5×PBS for 12 h and twice against 0.1×PBS for 12 h. The Ni²⁺-charged polymer is concentrated 10-fold in vacuo resulting in a 6% (w/v) solution. The size of Ni²⁺-charged poly(AAm-co-NTA-AAm) is analyzed by gel permeation chromatography on a Shodex OHpak SB-806 HQ (8.0 mm×300 mm, Showa Denko, Kawasaki, Japan) column using PBS as mobile phase at a flow rate of 0.5 ml/min (Waters 2796 Alliance Bio, Waters AG, Baden, Switzerland). Detection is performed at 280 nm and 390 nm using a Waters 2487 UV-detector. As size standards, poly(styrenesulfonic acid sodium salt) (Fluka, Buchs, Switzerland) is used.

### Example 6

### Hydrogel Formation and Characterization

Purified GyrB (80 mg/ml) in PBS is mixed with coumermycin (50 mg/ml in DMSO) at a molar ratio of GyrB:coumermycin=2:1 and incubated for 30 min at room temperature. Dimerized GyrB is subsequently added to 4.5 μl poly(AAm-co-NTA-AAM) (as 6% w/v solution in PBS) per mg GyrB and mixed by gently stirring. The hydrogel forms immediately and is incubated at 4° C. in a humidified atmosphere for 20 h prior to incubating the hydrogel for 12 h in PBS. For investigation of trigger-inducible hydrogel dissolution, the gel is incubated in PBS in the presence of different novobiocin (Fluka, cat. no. 74675) concentrations and the dissolution is monitored optically (GelJet Imager 2004, Intas, Göttingen, Germany) and by quantification of GyrB release into the buffer using the Bradford method. Error bars represent the standard deviation from three experiments.

### Example 7

### Hydrogel Formation and Characterization with Additional Crosslinks

Purified GyrB (80 mg/ml) in PBS is mixed with coumermycin (50 mg/ml in DMSO) at a molar ratio of GyrB:coumermycin=2:1 and incubated for 30 min at room temperature. Dimerized GyrB is subsequently incubated with an equimolar amount of dimethyl suberimidate and incubated at room temperature for 60 min. Dimerized and crosslinked GyrB is subsequently added to 4.5 μl poly(AAm-co-NTA-AAM) (as 6% w/v solution in PBS) per mg GyrB and mixed by gently stirring. The hydrogel forms immediately and is incubated at 4° C. in a humidified atmosphere for 20 h prior to incubating the hydrogel for 20 h in PBS or in PBS supplemented with 1 mM novobiocin. Swelling of the gel is monitored optically (FIG. 5). Error bars represent the standard deviation from three experiments.

### Example 8

### Construction of a Hydrogel Comprising a Polymer, a First Polypeptide and a Polypeptide Binding Partner where the Polypeptide Binding Partner is a Small Molecule

Synthesis of amino-functionalized novobiocin. Novobiocin is functionalized with an amino group by reacting novobiocin dissolved in DMF with K₂CO₃ and 2-(Boc-amino)ethyl bromide over night under reflux. The reaction mixture is concentrated in vacuo, the residue dissolved in dichloromethane with acetic acid and purified by column chromatography. The Boc-protected compound is dissolved in 50% TFA in dichloromethane for de-protection of the amine group. The solvents and the acid are evaporated.

Synthesis of an amine-reactive polymer. Acryloxysuccinimide is co-polymerized with acrylamide (molar ratio 1:4) in THF using AIBN as redox initiator. The resulting polymer (pAAm-succinimide) is precipitated and dried in vacuo.

Coupling of amino-functionalized novobiocin to the polymer. Amino-functionalized novobiocin is mixed with pAAM-succinimide (NH₂-Novobiocin:Succinimide=1.5:1, mol/mol) in PBS pH 8.0 and reacted over night at room temperature. The resulting polymer is dialyzed (MW cut-off: 3′500 Da) against water and lyophilized.

Construction of the hydrogel. Novobiocin-functionalized polyacrylamide dissolved in PBS is mixed with hexahistidine-tagged GyrB and poly(AAm-co-Ni²⁺-NTA-AAm) resulting in gelation. The polymer is swollen in PBS over night. Addition of increasing novobiocin concentrations to the hydrogel results in a dose-dependent dissolution of the gel.

### Example 9

### Construction of a Hydrogel Comprising a Polymer, a First Polypeptide and a Polypeptide Binding Partner where the Polypeptide Binding Partner is a Nucleic Acid

Synthesis of a DNA-functionalized polymer. Oligonucleotides encoding the tetO operator in sense and antisense orientation are synthesized, where the first oligo further contains an amino group at its 5′ end (NH₂). Both oligo strains are annealed, dissolved in PBS, pH 8.0 and mixed with succinimide-functionalized polyacrylamide (see Example 8) and incubated over night. The resulting DNA-functionalized polymer is subjected to ultrafiltration (MW cut-off 5′000 Da) and finally dissolved in PBS.

Production of hexahistidine-tagged TetR. The coding sequence for the tetracycline-repressor TetR is fused to a hexahistidine tag and expressed in E. coli BL21* (DE3) pLysS by IPTG induction. The protein is purified via Ni²⁺ affinity chromatography.

Construction of the Hydrogel. The tetO-functionalized polymer is mixed with hexahistidine-tagged TetR prior to the addition of poly(AAm-co-Ni²⁺-NTA-AAm). A hydrogel forms that can be dissolved by the addition of tetracycline antibiotics in the presence of Mg²⁺.

### Example 10

### Construction of a Hydrogel Comprising a Polymer, a First Polypeptide and a Polypeptide Binding Partner where the Polypeptide Binding Partner is a Second Polypeptide

The first polypeptide and the polypeptide binding partner is F_{M} (FKBP harbouring an F36M mutation). A polynucleic acid encoding F_{M} is fused with its 3′ end to a polynucleic acid encoding six histidine residues. The construct is cloned under the control of a T₇ promoter and expressed in E. coli BL21*. The cells are harvested by centrifugation, lysed in a French press and cell debris are eliminated by centrifugation. The cleared lysate is passed over a Ni²⁺-NTA column for affinity purification of hexahistidine-tagged F_{M}. F_{M} is eluted using a 300 mM imidazole-containing buffer and the buffer is exchanged to PBS by ultrafiltration. F_{M} is concentrated to 50 mg/ml by ultrafiltration. Purified and concentrated F_{M} is mixed with poly(AAm-co-NTA-AAM) (as 6% w/v solution in PBS, 10 μl poly(AAm-co-NTA-AAM) per 750 μg F_{M}). The hydrogel forms immediately and is incubated at 4° C. in a humidified atmosphere for 20 h prior to incubating the hydrogel for 12 h in PBS. For investigation of trigger-inducible hydrogel dissolution, the gel is incubated in PBS in the presence of different FK506 or rapalog concentrations and the dissolution is monitored optically (GelJet Imager 2004, Intas, Göttingen, Germany) and by quantification of F_{M} release into the buffer using the Bradford method.

### Example 11

### Construction of an F_{M}-Based Hydrogel Containing Additional Crosslinks

A hydrogel is constructed as described in Example 10 except that the F_{M} proteins are further stabilized by covalent bonds. Therefore, the concentrated F_{M} solution is incubated in the presence of 4 mol dimethylsuberimidate per mol F_{M} for 30 min at room temperature prior to mixing with poly(AAm-co-NTA-AAM). This hydrogel shows increased stability in cell culture media.

### Example 12

### Construction of a Hydrogel Based on Polyethylene Glycol and GyrB

The hydrogel consists of eight-arm polyethylene glycol coupled to GyrB which has been dimerized by coumermycin. Optionally, GyrB can further be crosslinked by dimethylsuberimidate. Therefore, GyrB incorporating a C-terminal cysteine is constructed by amplifying the gyrB gene using primers 5′-ggtacttgcacatatgtcgaattcttatgactcctccagtatc-3′ and 5′-ccagttacaagcttTCAGCAatggtgatggtgatgatgGCCTTCATAGTGGAAGTGGTCTTC-3′ and cloning it NdeI/HindIII into pWW301. GyrB-Cys protein is produced according to the protocol for GyrB described in Example 1. GyrB-Cys is reduced using TCEP (tris-carboxyethylphosphine) and coupled to 8-arm PEG carrying 8 terminal vinylsulfone groups according to a previous protocol (Rizzi S. C. and Hubbell J. A., Biomacromolecules 6, 1226-1238, 2005). PEG-coupled GyrB is dialyzed against PBS under reducing conditions (1 mM DTT) using a 100 kDa molecular weight cut-off to eliminate non-bound GyrB-Cys. PEG-coupled GyrB-Cys is concentrated to 80 mg/ml and mixed with coumermycin (50 mg/ml stock solution in DMSO, 1 mol coumermycin/2 mol GyrB). The forming hydrogels are incubated in a humid atmosphere for 24 h.

### Example 13

### Construction of a PEG-Based Hydrogel Containing Additional Crosslinks

A hydrogel as described in Example 12 is constructed except that the PEG-coupled GyrB solution is incubated with dimethylsuberimidate (DMS, 3 mol DMS per mol GyrB) for 30 min at room temperature prior to the addition of coumermycin. The resulting hydrogel shows higher stability in buffers than the hydrogel from Example 12. Addition of novobiocin triggers the dissolution of the hydrogel as quantified by measuring released protein in the swelling buffer using the Bradford method.

### Example 14

### PEG-Based Hydrogel Incorporating VEGF

A hydrogel as described in Example 13 is constructed except that VEGF (produced according to Example 2) is added to the GyrB prior to coupling to 8-arm PEG (molar ration VEGF:GyrB=1:24). Addition of novobiocin to the hydrogel results in a dose-dependent release of VEGF into the buffer as quantified by ELISA.

### Example 15

### Hydrogel Based on F_{M} Covalently Coupled to Polyacrylamide

F_{M} protein is produced as described in Example 10 and concentrated to 50 mg/ml. F_{M} is pegylated using succinimide-functionalized linear PEG (MW=5000 g/mol) at a molar ratio of Fₘ:PEG=2:1 for 30 min at room temperature. Subsequently, pegylated F_{M} is coupled to acryloxysuccinimide (molar ratio:acryloxysuccinimide:F_{M}=2:1), mixed with acrylamide (molar ratio:F_{M}:acrylamide=1:100) and polymerized by the addition of APS (3.6 μg/mg F_{M}) and TEMED (0.02 μl/mg F_{M}) over night at room temperature. The resulting hydrogels are equilibrated in PBS. Addition of FK506 results in the swelling of the hydrogels as monitored by gravimetric and optic analysis.

### Example 16

### Construction of a Hydrogel Comprising a Polymer, a First Polypeptide and a Polypeptide Binding Partner where the Polypeptide Binding Partner is a Small Molecule

Synthesis of a FKBP binding molecule. (S)-Pentyloxy-5-(N′-[4-(2-phenoxy-ethylamine)]-benzamidyl)-N-[2-oxo-2-(3,4,5-trimethoxyphenyl)acetyl)]proline is synthesized adapting a protocol from Siegal G., Overhand M. et al., Chem Med Chem 2, 1054-1070, 2007, as follows:

4-Acetoxy-N-[4-(hydroxy)phenyl]benzamide (1). A catalytic amount (a few drops) of N,N-dimethylformamide (DMF) is added to a solution of p-acetoxybenzoic acid (1 eq) in thionyl chloride (5 eq), and the solution is heated at reflux for 90 min. The mixture is cooled to room temperature, and excess thionyl chloride is removed under reduced pressure to furnish the acid chloride as a light-yellow oil. The acid chloride is dissolved in DMF and added slowly at a temperature of 0° C. to a solution of p-aminophenol (3 eq) and a catalytic amount of 4-dimethylaminopyridine (DMAP) in DMF. Stirring is continued for 1 h, after which the solution is transferred to a 2 L separatory funnel with EtOAc. The mixture is washed three times with 2 M HCl, once with 0.5 M NaHCO₃, and once with brine. The organic layer is dried over Na₂SO₄ and concentrated in vacuo. Compound 1 is obtained as a white solid.

4-Acetoxy-N-[4-(2-phenoxy-(Boc-ethylamine)]benzamide (2). Boc-aminoethyl bromide (1.25 eq) is added to a solution of compound 1 and K₂CO₃ (2 eq) in DMF. The reaction mixture is stirred at 120° C. over night. The solvent is removed in vacuo and the residue dissolved in EtOAc, washed with 1 M KHSO₄ and brine. The organic layer is dried over Na₂SO₄, concentrated in vacuo, and the remaining residue is purified by column chromatography.

4-Hydroxy-N-[4-(2-phenoxy-(Boc-ethylamine)]benzamide (3). NaOEt (1 eq) is added at room temperature to a solution of 2 (1 eq) in ethanol, and the solution is stirred until TLC indicates complete removal of the acetyl group (˜1 h). The solution is neutralized by addition of 2 M HCl, and the ethanol is removed under reduced pressure. The resulting suspension is dissolved in EtOAc and water (4:1 v/v) and transferred to a separatory funnel. The organic layer is separated and washed once with 2 M HCl, twice with 1 M NaHCO₃, and once with brine. The organic layer is dried over Na₂SO₄, concentrated in vacuo, and the remaining residue is purified by column chromatography to afford compound 3.

4-(5-(Benzyloxy)pentyloxy)-N-[4-(2-phenoxy-(Boc-ethylamine)]benzamide (4). Diethyl azodicarboxylate (2 eq, 40% in toluene) is added over a period of 10 min at 0° C. to a solution of compound 3 (1 eq), 5-(benzyloxy)pentan-1-ol (2 eq), and PPh₃ (2 eq) in THF. After 30 min the cooling bath is removed, and the solution is stirred at room temperature for 16 h. The solvent is concentrated in vacuo, and the remaining residue is purified by column chromatography. 4-(5-(Hydroxy)pentyloxy)-N-[4-(2-phenoxy-(Boc-ethylamine)]benzamide (5). Compound 4 is dissolved in a mixture of EtOAc and EtOH (1:1 v/v), and the solution is degassed by bubbling an argon stream through it for 2 min. Then Pd/C (10 wt % Pd on activated carbon) is added, the flask is equipped with a double-mantled hydrogen balloon, and the suspension is stirred for 16 h at room temperature. The catalyst is removed by filtering through Hyflo, and the mixture is concentrated in vacuo to afford the deprotected compounds in high purity.

(S)-tert-Butyl-N-(2-oxo-2-(3,4,5-trimethoxyphenyl)acetyl)proline (6). L-proline tert-butyl ester (1 eq) is added at 0° C. to a solution of 2-oxo-2-(3,4,5-trimethoxyphenyl)acetic acid (1.5 eq), EDC.HCl (1.5 eq), HOBt (2 eq), DIPEA (3 mmol), and a catalytic amount of DMAP in dichloromethane. After 30 min, the mixture is allowed to warm to room temperature, and stirring is continued for 16 h. The mixture is concentrated in vacuo, and the remaining residue is purified by column chromatography and provides 6.

N-(2-Oxo-2-(3,4,5-trimethoxyphenyl)acetyl)proline (7). TFA is added at room temperature to a solution of 6 in dichloromethane. The solution is stirred until TLC indicates complete removal of the tert-butyl group (˜8 h). 1 M NaHCO₃ is added slowly over a period of 10 min at room temperature. After gas formation has stopped, the mixture is transferred with EtOAc to a separatory funnel. The organic layer is discarded, and the aqueous layer is acidified carefully with 2 M HCl. The aqueous layer is extracted twice with EtOAc, and the organic layer is subsequently dried over Na₂SO₄ and concentrated in vacuo. Column chromatography provides 7.

(S)-Pentyloxy-5-(N′-[4-(2-phenoxy-(Boc-ethylamine)]benzamidyl)-N-[2-oxo-2-(3,4,5-trimethoxyphenyl)acetyl)]proline (8). 4-(5-(Hydroxy)pentyloxy)-N-[4-(2-phenoxy-(Boc-ethylamine)]benzamide 5 (1 eq) is added at a temperature of 0° C. to a solution of acid 7 (1.2 eq), EDC.HCl (1.2 eq), 1-hydroxybenzotriazole (HOBt, 1.5 eq), N,N-diisopropylethylamine (DIPEA, 1.2 eq), and a catalytic amount of DMAP in absolute dichloromethane. After 30 min, the mixture is allowed to warm to room temperature, and stirring is continued for 16 h. The mixture is concentrated in vacuo and purified by column chromatography affording compound 8.

(S)-Pentyloxy-5-(N′-[4-(2-phenoxy-ethylamine)]benzamidyl)-N-[2-oxo-2-(3,4,5-trimethoxyphenyl)acetyl)]proline (9). TFA (10 mmol) is added at room temperature to a solution of compound 8 to be deprotected in dichloromethane, and the solution is stirred for 2 h (TLC control). 1 M NaHCO₃ is added slowly over a period of 10 min at room temperature. After the gas formation has stopped, the mixture is transferred with EtOAc to a separatory funnel. The organic layer is extracted once with 1M NaHCO₃, and dried over Na₂SO₄. The organic layer is concentrated in vacuo. The desired compound 9 is purified by column chromatography.

Synthesis of an amine-reactive polymer. Acryloxysuccinimide is co-polymerized with acrylamide (molar ratio 1:4) in THF using AIBN as redox initiator. The resulting polymer (pAAm-succinimide) is precipitated and dried in vacuo.

Coupling of amino-containing FKBP binding molecule to the polymer. Compound 9 is mixed with pAAM-succinimide (compound 9:succinimide=1.5:1, mol/mol) in PBS pH 8.0 and reacted over night at room temperature. The resulting polymer is dialyzed (MW cut-of: 3′500 Da) against water and lyophilized.

Construction of the hydrogel. FK506-analog-functionalized polyacrylamide dissolved in PBS is mixed with hexahistidine-tagged FKBP and poly(AAm-co-Ni2+-NTA-AAm) resulting in gelation. The polymer is swollen in PBS over night. Addition of increasing FK506 concentrations to the hydrogel results in a dose-dependent dissolution of the gel.

1. A hydrogel comprising a polymer selected from poly-vinyl-based polymers, polypeptides and polycarbohydrates, a first polypeptide and a polypeptide binding partner, wherein the first polypeptide and the polypeptide binding partner are selected from the group consisting of GyrB-GyrB, FKBP-FRB, F_{M}-F_{M}, ToxT-ToxT, DHFR-DHFR, FKBP-FKBP, FKBP-Cyp, Cyp-Cyp, E-ETR, PIP-PIR, TetR-tetO, ArgR-argO, ArsR-arsO, HucR-hucO, GyrB-aminocoumarin antibiotic, FKBP-mTOR inhibitor, FRB-mTOR-inhibitor, F_{M}-mTOR inhibitor, Cyp-cyclosporin, Cyp-ascomycin, DHFR-antifolate, streptavidin-biotin analog, avidin-biotin analog, neutravidin-biotin analog, steroid hormone receptor-steroid hormone, and ToxT-virstatin, indicated as pairs of first polypeptide-polypeptide binding partner, wherein the first polypeptide and/or the polypeptide binding partner are covalently linked to the polymer or linked to the polymer by a strong, specific non-covalent linkage, and wherein the interaction between the first polypeptide and the polypeptide binding partner is non-covalent, stabilizes the hydrogel and is cleaved by the addition or withdrawal of a modulating compound.

2. The hydrogel of claim 1, wherein the first polypeptide is linked to the polymer and the polypeptide binding partner is linked to a compound of interest.

3. The hydrogel of claim 1, wherein the first polypeptide is linked to a compound of interest and the polypeptide binding partner is linked to the polymer.

4. The hydrogel of claim 1 further comprising a compound of interest physically entrapped in the hydrogel or linked to the polymer.

5. The hydrogel of claim 2, wherein the compound of interest is a drug.

6. (canceled)

7. The hydrogel of claim 1, wherein the polymer is selected from polyacrylamide, polyethylene glycol, poly-dimethyl-diallyl-ammonium chloride, N-(2-hydroxypropyl)methacrylamide, fibrin, collagen, poly-L-lysine, alginate, celluloses, dextran, and starch.

8. (canceled)

9. The hydrogel of claim 1, wherein the first polypeptide and the polypeptide binding partner are selected from the group consisting of GyrB-GyrB, FKBP-FRB, F_{M}-F_{M}, ToxT-ToxT, DHFR-DHFR, FKBP-FKBP, FKBP-Cyp, Cyp-Cyp, E-ETR, PIP-PIR, TetR-tetO, ArgR-argO, ArsR-arsO, or HucR-hucO, indicated as pairs of first polypeptide-polypeptide binding partner.

10. The hydrogel of claim 1, wherein the first polypeptide and the polypeptide binding partner are one and the same polypeptide having a tendency to dimerize by addition or withdrawal of the modulating compound.

11. The hydrogel of claim 10, wherein the polypeptide having a tendency to dimerize is selected from GyrB, F_{M}, ToxT, DHFR, FKBP, and Cyp.

12. The hydrogel of claim 1, wherein the first polypeptide and/or the polypeptide binding partner are covalently linked to a further polymer influencing solubility.

13. The hydrogel of claim 1, wherein the first polypeptide and/or the polypeptide binding partner are homomultimers or heteromultimers with other polypeptides or polypeptide-binding partners.

14. A system of drug delivery comprising the hydrogel of claim 1, wherein either the first polypeptide or the polypeptide binding partner are linked to the polymer, and the corresponding polypeptide binding partner or the first polypeptide, respectively, are linked to a drug, and further comprising a compound cleaving the interaction between the first polypeptide and the polypeptide binding partner.

15. A system of drug delivery comprising the hydrogel of claim 1, wherein the first polypeptide and/or the polypeptide binding partner are linked to the polymer and a drug is physically entrapped in the hydrogel structure or bound to the polymer stabilized by the interaction between polypeptide and the polypeptide binding partner, and further comprising a compound cleaving the interaction between the first polypeptide and the polypeptide binding partner.

16. The hydrogel of claim 10, wherein the polymer is polyethylene glycol or polyacrylamide, and the polypeptide having a tendency to dimerize is F_{M} covalently linked to said polymer.

17. The hydrogel of claim 3, wherein the compound of interest is a drug.

18. The hydrogel of claim 4, wherein the compound of interest is a drug.


SEQUENCE LISTING (3 sequences)

SEQ ID NO: 1 - DNA, 43 bp, Artificial
  Synthetic construct, GyrB primer
ggtacttgcacatatgtcgaattcttatgactcctccagtatc

SEQ ID NO: 2 - DNA, 44 bp, Artificial
  Synthetic construct, GyrB primer
ccagttacaagcttatggtgatggtgatgatggccttcatagtg

SEQ ID NO: 3 - DNA, 62 bp, Artificial
  Synthetic construct, GyrB primer
ccagttacaagctttcagcaatggtgatggtgatgatggccttcatagtggaagtggtcttc